(19) United States
(12) Patent Application Publication
(10) Pub. No.: US 2023/0170052 A1
(43) Pub. Date: Jun. 1, 2023

(54) METHOD AND SYSTEM FOR PROCESSING GENOMIC DATA

(71) Applicant: The University of Sydney, New South Wales (AU)

(72) Inventor: Zac Chatterton, New South Wales (AU)

(21) Appl. No.: 17/921,769

(22) PCT Filed: Apr. 30, 2021
(86) PCT No.: PCT/AU2021/050390

§ 371 Date: Oct. 27, 2022

(30) Foreign Application Priority Data
Apr. 30, 2020 (AU) 2020901357

(51) Int. Cl. G16B 50/30; G16B 30/00; G16B 20/00

(52) U.S. Cl. CPC G16B 50/30; G16B 30/00; G16B 20/00

(57) ABSTRACT

The present invention is concerned with computer-implemented methods, systems and software products for processing nucleic acid sequence data, the data including information on the methylation status of cytosine residues present in the sequence data, the method comprises the steps of assigning a binary methylation value selected from 5 one of two possible values, to one or more of the cytosine residues; extracting k-mers from the sequence data, each k-mer including a cytosine residue having one of the methylation values; and storing the k-mers in a database.

### FIELD OF THE INVENTION

The present invention broadly relates to epigenetics, systems for analysis of genomic data and diagnostics. More specifically, the invention relates to a method for processing genomic data to enable identification of the cell of origin from which a sample nucleic acid sequence originates.

### BACKGROUND OF THE INVENTION

Unlike traditional biopsies involving invasive surgery, so-called “liquid biopsies” utilise samples of blood or other extracellular biological fluid that can be obtained with minimal invasiveness. Such samples often contain cell-free DNA (cfDNA). Liquid biopsy techniques exploit genetic differences between the subject from which the sample was taken and “foreign” DNA present in the sample. For example, some prenatal diagnoses techniques utilise the phenomenon of cell-free foetal DNA circulating in the mother's bloodstream. Liquid biopsies can also be used to monitor the progress of a transplanted organ that incorporates “foreign” DNA.

However, using genetic information to diagnose certain conditions, such as neurodegenerative, inflammatory or ischemic diseases, is problematic as the conditions do not result in nucleotide changes in the host DNA. For example, during cirrhosis of the liver, genomic DNA from the affected liver cells does not differ from that of host cells. Similarly, DNA from affected brain cells does not differ from the affected host cell during neurodegeneration observed in dementia.

It would be advantageous to develop techniques that could assist in diagnosing diseases that are not characterised by nucleotide changes in the host DNA.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

According to a first aspect of the present invention there is provided a computer-implemented method for processing nucleic acid sequence data, the data including information on the methylation status of cytosine residues present in the sequence data, the method comprising the steps of:

assigning a binary methylation value selected from one of two possible values, to one or more of the cytosine residues;

extracting k-mers from the sequence data, each k-mer including a cytosine residue having one of the methylation values; and

storing the k-mers in a database.

The present invention encompasses a technique for processing nucleic acid sequence data to create binary references of tissue-specific epigenetic information in the form of cell-of-origin DNA methylation patterns. The binary references are encapsulated in k-mers which can be beneficially stored in a database for later querying.

The present invention also provides a method for efficiently constructing a database of DNA methylation k-mers. Such a database can be suitably queried to determine the cell-of-origin of an individual sequencing read extracted from a biological sample. In turn, information about the cell-of-origin can be utilised to diagnose diseases attributed to cell-death within an organ system. Importantly, cell-death can be detected in the absence of nucleotide changes in the host DNA.

Preferably, the database includes a hash table and the method includes the step of mapping each k-mer to a respective hash code, each hash code being suitable to address a storage location in the database. Indexing a hash table with k-mers that encapsulate DNA methylation information allows for efficient querying of the database using k-mers extracted from a biological sample.

Preferably, the assigning step is performed by reference to a threshold. According to some embodiments, the threshold is modified according to the cell-of-origin of the nucleic acid sequence data.

This combination of binarization, k-mer lookup and co-methylation thresholding realise a rapid and accurate deconvolution strategy for tissue or cell-specific DNA within a heterogenous mixture of DNA, such as cfDNA.

According to another aspect of the present invention, there is provided a computer-readable storage medium having instructions encoded thereon which when executed by a processor, cause the processor to process nucleic acid sequence data, the data including information on the methylation status of cytosine residues present in the sequence data, by:

assigning a binary methylation value selected from one of two possible values, to one or more of the cytosine residues;

extracting k-mers from the sequence data, each k-mer including a cytosine residue having one of the methylation values; and

storing the k-mers in a database.

According to another aspect of the present invention, there is provided a method of generating a library of polynucleotide subsequences representative of one or more cells of origin, the method comprising:

a) providing a plurality of DNA methylation profiles for a genomic sequence for a cell of origin;

b) annotating the genome sequence of the cell of origin at each cytosine residue, based on the DNA methylation profiles, to create binarised reference genomic information for the cell of origin;

c) repeating steps a) and b) for one or more additional cells of origin;

d) generating a series of subsequences of length k (k-mers) from the binarised reference genomic information from the different cells of origin;

thereby generating a library of polynucleotide subsequences representative of one or more cells of origin.

Preferably, the step of annotating the genome of the cell of origin comprises i) binarising each cytosine nucleotide in the genome and ii) inserting the binary value assigned to each cytosine during the binarisation into the genomic context of the cytosine nucleotide, to create sense and antisense genomic DNA fragments comprising information on the methylation status of each cytosine nucleotide.

The binary values may comprise: “methylated” or “not methylated”; “C” or “T”, or two other values representing the methylation state of each cytosine.

In any embodiment, the binary value is based on a threshold such that when the cytosine is methylated in at least 50% of DNA methylation profiles, the cytosine nucleotide is designated ‘methylated’ (or C) for the purpose of creating the binarised reference and wherein when the cytosine is methylated in less than 50% of DNA methylation profiles, the cytosine nucleotide is designated ‘not methylated’ (or T) for the purpose of creating the binarised reference.

The step of inserting the binarised cytosines into the genomic context may be performed using GATK_FastaAlternateReferenceMaker software such that the binarised reference genomic information is obtained in FASTA format.

The binarised reference genomic information may be prepared using a suitable software package. To some extent, the choice of software package is motivated by the process that was used to generate k-mers from a biological sample (such as cfDNA). Where bisulfite sequencing is used, the sense and antisense strand of the reference genome may be prepared using bismark_genome-preparation software. In this regard, preparing the sense and antisense strands produces an in-silico bisulphite-converted reference genome. The software routines take a human genome sequence as input and convert all of the Cs to Ts (working on the assumption that all cytosines are unmethylated). The conversion process is similar to inserting binarised cyotsines into the genomic context, but with the difference that there is no prior knowledge of the methylation status of cells-of-interest. Preparation of sense and antisense strands is necessary for bisulphite sequencing, but not necessarily for other techniques such as TAPS.

Typically, the method further comprises grouping of a plurality of binarised reference genomic information subsets in FASTA format such that the grouped information can be utilised for k-mer indexing. In certain examples, the grouping of subsets may be performed using suitable genomic feature analysis libraries, such as the Bedtools package.

Optionally, the method further comprises the step e) of indexing the k-mers in order to identify counts of k-mers that are associated with each cell of origin. Preferably the indexing comprises identifying counts of k-mers that are unique for each cell of origin wherein the counts refer to the number of k-mers uniquely assigned to a given cell of origin. In preferred embodiments, the k-mers are indexed from contiguous genomic regions within the binarised reference genomic information for each cell of origin.

In any embodiment, each k-mer has a nucleotide sequence length of at least 10 nucleotides, at least 20 nucleotides, at least 30 nucleotides or more.

In any embodiment the method comprises the step f) of storing the subsequences in a database.

In any embodiment the DNA methylation profiles are derived from whole genome bisulfite sequencing data, however other techniques (preferably those with single-base pair resolution) can be used such, such TET-assisted pyridine borane sequencing (TAPS), Third Generation Sequencing (long read sequencing) or similar. When whole genome bisulfite sequencing data is used, the DNA methylation profiles are preferably derived from NGS analysis following bisulfite treatment.

In further embodiments, the indexed k-mers are utilised for determining the cell of origin for a sequence of unknown origin. Preferably the sequence of unknown origin is obtained from a sample of heterogeneous DNA; the sequence may be obtained from NGS (or other suitable techniques), wherein each NGS read of the sample is queried against the indexed k-mers; alternatively, the sequence may be targeted DNA methylation sequencing (bisulfite sequencing) that comprises sequence from only a fragment of the genome; the heterogeneous DNA population may be from a cfDNA sample, for example obtained from an extracellular biological fluid.

According to another aspect of the present invention, there is provided a computer-implemented method of processing genome sequence data for a cell of origin, the method comprising:

a) annotating the genome sequence data at each cytosine residue, based on a DNA methylation profile, to create binarised reference genomic information for the cell of origin;

b) repeating step a) for one or more additional cells of origin; and

c) generating a series of subsequences of length k (k-mers) from the binarised reference genomic information from the different cells of origin to thereby generate a library of polynucleotide subsequences representative of one or more cells of origin.

According to another aspect of the present invention there is provided a computer-readable storage medium having instructions encoded thereon which when executed by a processor, cause the processor to process genome sequence data for a cell of origin, by:

a) annotating the genome sequence data at each cytosine residue, based on a DNA methylation profile, to create binarised reference genomic information for the cell of origin;

b) repeating step a) for one or more additional cells of origin; and

c) generating a series of subsequences of length k (k-mers) from the binarised reference genomic information from the different cells of origin to thereby generate a library of polynucleotide subsequences representative of one or more cells of origin.

In a further aspect, the present invention provides a method of determining a tissue or cell of origin of a nucleic acid of unknown origin, the method comprising:

providing or having obtained a library or database of subsequences representative of one or more cells of origin, according to the methods described herein;

providing or having obtained a nucleic acid sequence of unknown origin for which a cell of origin is to be determined;

querying subsequences of the nucleic acid sequence against the database of subsequences,

thereby determining the tissue or cell of origin of the nucleic acid.

Preferably, the library includes a hash table and the method includes the step of mapping each subsequence to a respective hash code, each hash code being suitable to address a storage location in the database.

Hash table look up can be completed using any suitable software program known to the skilled person, including but not limited to Kallisto.

Typically, the nucleic acid sequence of unknown origin is obtained from a sample of heterogeneous DNA. The heterogenous DNA population is preferably from a cfDNA sample, for example obtained from an extracellular biological fluid. The cfDNA may be obtained from blood plasma, from cerebrospinal fluid (CSF), from saliva, from lacrimal fluid or any other extracellular biological fluid comprising cfDNA.

The sequence may be obtained from NGS (or other suitable technique), wherein each NGS read of the sample is queried against the indexed k-mers; alternatively, the sequence may be targeted DNA methylation sequencing (bisulfite sequencing) that comprises sequence from only a fragment of the genome.

The present invention also provides a method of characterizing a cfDNA sample from a subject, comprising:
receiving a plurality of sequencing reads for a cfDNA sample from a subject, wherein each sequencing read comprises methylation sequencing data obtained from a consecutive nucleic acid sequence of 25 or more nucleic acids; and
comparing the plurality of sequencing reads to the subsequences of a library or database described herein to compute one or more likelihood scores, wherein the likelihood score is indicative of the likelihood that the sequences in the cfDNA sample correspond to sequences from a given cell-of-origin.

According to a further aspect of the present invention, there is provided a method of determining the likelihood that an individual is suffering from a disease or condition characterised by cell death in an organ or cell of interest, the method comprising:

providing an individual for whom diagnosis of a disease or condition characterised by cell death in an organ or cell of interest is required;

providing or having obtained a query nucleic acid sequence of unknown origin, obtained from a sample of plasma-derived cfDNA from the individual;

providing of having obtained a database of subsequences representative of one or more cells of origin according to the methods described herein, wherein the one or more cells of origin comprises cells of one or more organs, organ tissues or cell of interest;

determining that the individual is likely suffering from a condition or disease characterised by cell death in an organ when subsequences of the query nucleic acid coincide with subsequences in the database representative of cells of an organ, organ tissues or cell of interest; or

determining that the individual is likely not suffering from a condition or disease characterised by cell death in an organ when one or more subsequences of the query nucleic acid sequence do not coincide with subsequences in the database representative of cells of an organ, organ tissues or cell of interest.

In any embodiment, the disease or condition characterised by cell death in an organ or tissue may be a neurodegenerative disorder (wherein the disorder is characterised by death of neurological cells), a disorder of the thyroid gland (wherein the disorder is characterised by death of thyroid follicular cells or parafollicular cells or a kidney disorder (wherein the disorder is characterised by death of renal cells.

In any embodiment, the query nucleic acid is from a cell or tissue.

In some embodiments, the query nucleic acid is from a cell type selected from the group consisting of a pancreatic beta cell, a pancreatic exocrine cell, a hepatocyte, a brain cell, a lung cell, a uterus cell, a kidney cell, a breast cell, an adipocyte, a colon cell, a rectum cell, a cardiomyocyte, a skeletal muscle cell, a prostate cell and a thyroid cell.

In some embodiments, query nucleic acid is from a tissue selected from the group consisting of pancreatic tissue, liver tissue, lung tissue, brain tissue, uterus tissue, renal tissue, breast tissue, fat, colon tissue, rectum tissue, heart tissue, skeletal muscle tissue, prostate tissue and thyroid tissue.

In some embodiments, the query nucleic acid is from a cancer cell, tumour cell or transformed cell.

In some embodiments, the query nucleic acid is from a foreign donor, wherein the foreign donor may be providing a blood transfusion, donor transplant or graft.

In some embodiments, the query nucleic acid is from a eukaryotic or prokaryotic organism.

In some embodiments, the query nucleic acid is from a virus.

The disease or condition characterised by cell death extends to:
infection as inferred by the detection of bacterial, viral, and/or parasitic nucleic acid sequences;
inflammation as inferred by the detection of blood-cell derived nucleic acids;
degeneration as inferred by the detection of cells of the human body;
organ transplant monitoring as inferred by the detection/absence of nucleic acids from the implanted organ;
stem cell monitoring as inferred by the detection/absence of nucleic acids from transplanted stem cells; and
dietary requirements/deficiencies as inferred by the detection/absence of nucleic acids from nutrient sources such as fruit, vegetable, grains and animal-derived foodstuffs.

According to another aspect of the present invention, there is provided a method of determining the likelihood that an individual is suffering from a neurodegenerative disease or condition, the method comprising:

providing an individual for whom diagnosis of a neurodegenerative disease or condition is required;

providing or having obtained a query nucleic acid sequence of unknown origin, obtained from a sample of plasma-derived cfDNA from the individual;

providing or having obtained a database of subsequences representative of one or more cells of origin according to the methods described herein, wherein the one or more cells of origin comprises cells of neurological origin;

determining that the individual is likely suffering from a neurodegenerative disease or condition when one or more subsequences of the query nucleic acid sequence coincide with subsequences in the database representative of cells of neurological origin; or

determining that the individual is likely not suffering from a neurodegenerative disease or condition when one or more subsequences of the query nucleic acid sequence do not coincide with subsequences in the database representative of cells of neuronal origin.

In a further aspect, there is provided a method of detecting the presence of DNA from a cell or tissue of origin and identifying if a subject has a disease or condition characterised by necrosis, apoptosis or other mode of death of the cell or tissue, the method comprising:

receiving sequencing data of cell-free methylated DNA from a test sample obtained from a subject suspected of having, or at risk of having a disease or condition characterised by necrosis, apoptosis or other mode of death of the cell or tissue;

comparing subsequences of the cell-free methylated DNA to a library or database as described herein;

identifying that the subject has a disease or condition characterised by necrosis, apoptosis or other mode of death of the cell or tissue when one or more compared subsequences coincide with subsequences present in the library or database; or

identifying that the subject does not have a disease or condition characterised by necrosis, apoptosis or other mode of death of the cell or tissue when one or more compared subsequences do not coincide with subsequences present in the library or database.

In an aspect, there is provided a computer-implemented method of determining the cell or tissue of origin for cfDNA obtained from a subject, the method comprising:

receiving, at at least one processor, sequencing data of cell-free methylated DNA from a subject sample;

comparing, at the at least one processor, subsequences of the sequencing data to control cell-free methylated DNA subsequences from healthy and cancerous individuals;

identifying, at the at least one processor, that one or more of the compared subsequences coincides with one or more of the cancerous cell-free methylated DNA subsequences comprised in the control cell-free methylated DNA subsequences.

In another aspect, there is provided a computer program product for use in conjunction with a general-purpose computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

In an aspect, there is provided a computer readable medium having stored thereon a data structure for storing the computer program product described herein.

In broad terms, the present invention involves defining DNA methylation k-mers that are specific to particular tissues or cells-of-interest and quantifying bisulfite (or other methylation-inducing treatments) sequencing reads derived from these tissues/cells-of-interest within a heterogenous mixture.

The present invention, at least in preferred embodiments:
binarizes DNA methylation to define a tissue or cell DNA methylation pattern that can be used for k-mer based lookups;
incorporates co-methylation thresholds to define the tissue or cells-of-origin of DNA within a heterogenous mixture of DNA.

The binarization of a DNA methylation fractional signal and subsequent mapping into the genomic context also limits the effects of technical artefacts and biological contamination.

In summary, the present invention makes use of a new approach to analysis of cfDNA and provides methods for the diagnosis of disease based on analysis of DNA methylation profiles and use thereof to determine tissue/cell-of-origin for cfDNA.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

An embodiment of the present invention is described by reference to the following drawings in which:

FIG. 1 is a graph illustrating the distribution of DNA methylation markers in fluorescence-activated cell separated Central Nervous System neurons (neun+, GSM1173776), single-cell collapsed cell-cluster of human deep layer 1 neurons, and a single hDL-1 cell (GSM2558762).

FIG. 2 is a schematic illustration of the process of binarizing methylated cytosine residues and inserting the result into genomic context (BINGO).

FIG. 3 is a schematic illustration of the process of indexing DNA methylation k-mers extracted from the genomic context of a cell-of-origin.

FIG. 4 is a schematic illustration of the process of assigning a cell-of-origin to a DNA molecule.

FIG. 5 shows two graphs of unique whole-genome-bisulphate-sequencing reads of cfDNA from CSF and plasma uniquely pseudoaligned (assigned) to Central Nervous System (CNS) cells; neurons (NeuN+) and Glia (NeuN−) [left], or blood cells [right].

FIG. 6 illustrates the results of investigations into the presence of brain-derived cfDNA and cortical volume changes.

FIG. 7 is a block diagram of a computer processing system configurable to perform various features of the present invention

### DETAILED DESCRIPTION OF THE EMBODIMENTS

### General

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

It will be understood that the invention disclosed and defined in this specification extends to all alternative combinations of two or more of the individual features mentioned or evident from the text or drawings. All of these different combinations constitute various alternative aspects of the invention.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in bioinformatics and genome analysis).

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

### Selected Definitions

As used herein, the term “DNA binarization” or “binarization of DNA methylation” refers to the programmatic assignment of a binary methylation value to a cytosine nucleotide. The two possible methylation values can comprise “methylated” and “unmethylated”; “C” and “T” or any other convenient representation.

The process of DNA binarization can also be seen as annotating DNA methylation as either “methylated” or “un-methylated”. DNA methylation can, for example, result from bisulfite sequencing or TET-assisted pyridine borane sequencing (TAPS). The binarization of DNA methylation allows a DNA methylation signal to be programmatically annotated within the genomic context of the nucleotide sequence.

The term “fractional methylation” or “FM” refers to a fractional representation of DNA methylation at an individual cytosine, whereby 1 represents fully methylated and 0 represents not methylated.

The term “k-mer” refers to a set of subsequences, of fixed length k, that are contained within a biological sequence. Typically, a k-mer refers to all of a sequence's subsequences of length k. In this specification, the term ‘k’ is used both in respect of the length of a k-mer and to define a set of hash functions. The correct use of ‘k’ will be clear from the context.

The phrase “k-mer indexing” refers to creating an index which includes an entry for each k-mer, with each entry storing the location/s of the k-mer within the biological sequence.

The phrase, “k-mer lookup” refers to an operation querying a k-mer index with a specific k-mer to return location/s within the biological sequence where the specific k-mer is found. A k-mer lookup operation can return an error value such as an ‘indeterminate error’. A k-mer lookup can involve a series of lookup operations, wherein individual results are combined into a result summary.

cfDNA refers to DNA that is found in extracellular biological fluid (such as blood or a blood fraction) of an individual. “Circulating” is an art-recognised term, and refers to a substance (such as cfDNA) present in, detected or identified in, or isolated from, a circulatory system of an individual, such as the blood system or lymphatic system. In particular, when cfDNA is “circulating” it is not located in a cell, and hence may be present in extracellular biological fluid such as blood plasma or serum or lymphatic fluid.

The term “condition” refers to a disruption of or interference with normal function, and is not to be limited to any specific condition, and will include diseases or disorders.

### DNA Methylation

DNA methylation is an epigenetic modification that is precisely and intricately involved in cellular function and specification of mammalian cells. The presence of a cytosine methylation at a given site is enough to block binding of factors to the DNA and to inhibit transcription. DNA methylation of single cytosines is typically represented as fractional measurements. For example, following bisulfite treatment (Frommer M et al., (1992) PNAS, 89(5): 1827-31) and Next Generation Sequencing (NGS) analysis or similar, the methylation of a cytosine can be characterised as C_{reads}/(C_{reads}+T_{reads}). Similarly, following microarray and qPCR interrogation the methylation of a cytosine can be characterised as C_{flouresence}/(C_{fluoresence}+T_{flouresence}).

Bisulfite conversion of DNA followed by NGS allows a DNA methylation signal to be analysed at a single molecule level. Whole-genome bisulfite sequencing (WGBS) generates cytosine (C) and thymine (T) nucleotides depending on whether the DNA is methylated at the particular cytosine base. The WGBS read is usually aligned to a reference genome and the number of reads with C and T nucleotides mapped to each cytosine in the genome can be summarised as a read-count table.

Whole-genome bisulfite sequencing (WGBS) reads often contain technical errors such as PCR artefacts, sequencing errors and bisulfite conversion failures. In addition, WGBS reads contain biological contamination derived from non-cell-of-interest (COI) cell-types contributing to the nucleotide pool. Fractional measurements of DNA methylation derived from multiple WGBS data covering a cytosine of interest reduce the impact of such errors and improves the accuracy of the DNA methylation quantification at individual cytosines for sequences from a cell-of-interest.

The fractional DNA methylation level can be determined at the level of each cytosine as a given position in the genome, which is computed as the ratio of the number of cytosine reads to the number of total reads. This measure is commonly used to quantify methylation levels of a specific cytosine.

### Thresholding and Binarization

The binarization of DNA methylation sites involves the programmatic annotation of DNA methylation as either C (methylated) or T (unmethylated), or annotated as C (unmethylated) or T (methylated). The computational steps to achieve binarization involve:

1) thresholding of DNA methylation fractional measurements at individual cytosines, whereby a threshold level is established to assign a residue as either methylated or unmethylated. The threholding procedure may need to account for the sequencing technique used; and

2) annotation of the reference genome to reflect the binarized DNA methylation signal.

The binarized DNA methylation signal is inserted into genomic-context (BINGO). Insertions are performed recursively for each COI, thereby establishing a unique DNA methylation reference genome for each COI.

Bisulfite conversion of DNA results in non-methylated cytosines being converted to thymine. This process renders two non-complementary DNA strands (top and bottom, represented by CT/GA). Binarized DNA methylation signals (above) are mapped to their respective genomic context for both the CT/GA strands. Genome preparation and methylation signal mapping can be performed in silico using suitable routines from genomic analysis software such as bismark and GATK.

The DNA methylation binaries within the genomic context of each COI can be conveniently stored as FASTA files for each CT/GA strand. The grouping of multiple COI DNA methylation binaries within the genomic context can then be performed (for example using bedtools) for genomic regions with coverage between all COI. FASTA files combining all COI and both CT/GA strands can be produced using analogous techniques. The FASTA file can then be utilised for k-mer indexing and hash table lookup using several software tools including Kallisto.

### K-mer Indexing

The techniques of the present invention can be used to ‘deconvolute’ NGS data in which single-sequencing reads are assigned to a cell-of-origin.

DNA methylation k-mers may be generated using a variety of available software. Examples of such software include Kallisto and Kraken Unique. The k-mers that are indexed are unique and can be overlapping. The k-mer lengths can be adjusted depending on the analytical software used, for example k-mers of length of 31 can be used with Kallisto for k-mer indexing.

Cell-type specific DNA methylation patterns are analogous to gene sequences that delineate transcripts. The gene sequence substrings (i.e k-mers) can delineate between transcripts, however, bisulfite sequencing k-mers are unique between DNA strands exhibiting cell-specific DNA methylation patterns within a pooled mixture of DNA.

The output of the exemplified k-mer indexing process is DNA fragments (e.g. bisulfite sequencing reads) that have been assigned to each cell-of-origin.

The whole sequence of each NGS read can readily be queried against k-mers indexed using the techniques of the present invention. The indexed k-mers against which queries are run, are assay dependent. For example, when performing whole-genome DNA methylation sequencing experiments (such as WGBS) on a patient's cfDNA, k-mers can be indexed across the whole genome of multiple COI's. Targeted DNA methylation sequencing assays (bisulfite sequencing) that focus on only a few regions of the genome within a patient's cfDNA can also be used. For such an assay, k-mers are indexed only within genomic regions targeted by the assay of multiple COI's.

### Detection of DNA Derived from cfDNA

The cell-specificity of DNA methylation patterns in a single molecule (such as from an NGS read), potentially allows the tissue/cell-of-origin (COO) of a fragment of cfDNA to be determined.

The determination of cell-of-origin (COO) of cfDNA, for example cfDNA found in a sample of plasma obtained from a patient, has particular utility in determining whether cell death from a particular tissue is occurring. For example, the presence of cfDNA of neuronal origin in a plasma sample may be indicative of a neurodegenerative disease characterised by the death of neuronal cells. In this regard, an increased presence of such cells in the plasma can be detected by determining that cfDNA in the plasma originated from neuronal cells.

The COO data can result in the assignment of DNA fragments following bisulfite sequencing of heterogeneous DNA mixtures such as a cfDNA mixture from a blood plasma sample. For instance, within a sample, the resulting counts of reads assigned to a cell of origin represent measurements of tissue/cell death with generalised diagnostic utility. For example, the present invention shows that cfDNA derived from cerebral spinal fluid (CSF) is enriched for DNA derived from the Central Nervous System, compared to cfDNA derived from peripheral blood plasma. This thereby provides a technique capable of detecting brain-cell death that can be used for the identification of various neurodegenerative diseases and/or provides a mechanism for detection of somatic DNA mutations that may arise in CNS cells.

For example, the detection of k-mers specific to neuronal bisulfite converted DNA derived from the brain, establishes a minimally invasive method for the detection or diagnosis of neurodegenerative disease through the blood. The applications could be naturally extended to any tissue or cell of the human body i.e. the detection of liver-derived DNA within cfDNA to detect cirrhosis of the liver.

### Detection of DNA Derived from a Pathogen

The present invention can be utilised to analyse bisulfite sequencing data from cfDNA for the detection of DNA derived from any pathogen, which would indicate an acute or chronic infection. A non-limiting example includes the detection of k-mers specific for Listeria monocytogenes bisulphate DNA. to identify Listeriosis. In combination with k-mers specific to brain-derived bisulfite DNA, the techniques could be used to test for encephalitis caused by Listeriosis.

### EXAMPLES

### Example 1—Process of Binarisation of DNA Methylation and Assigning cfDNA to a Cell-of-Origin

Publicly available whole genome DNA methylation profiles of 11 COIs were used. COls were selected with relevance to plasma and cerebral spinal fluid (CSF) derived (cfDNA) and included B-cell, CD14+ monocyte, CD34+ common myeloid progenitor, H1 and HUES64 ESC's, Natural Killer Cell, Spinal Cord, T-cell and Thyroid Gland, produced by the ENCODE consortium using WGBS (ENCSR284TCU, ENCSR017BUL, ENCSR388RMS, ENCFF601NBW, ENCSR354DMU, ENCSR334LSM, ENCSR334LSM, ENCSR458MAV, ENCSR663MXB, ENCSR601MHU). In addition, whole genome DNA methylation profiles from primary cortical Neurons (NeuN+) and Glia (NeuN−) produced by Lister and colleagues using WGBS (Lister R et al. (2013) Science, 341(6146): 1237905) were used.

Fractional DNA methylation levels from WGBS analysis of 11 the COI's in .bed file format were used. Independent .bed files from the multiple samples of a COI were combined using the bedtools routine unionbedg. Combined coverage and methylation were calculated and low-quality DNA methylation fractions (<5× coverage) were removed using a suitable utility. Strand information for each cytosine was obtained using the bedtools routine getfasta.

DNA methylation fractions were binarized using a fractional DNA methylation threshold of 0.5 (DNA methylation fractions>=0.5 were assigned C, DNA methylation fractions<0.5 were assigned T) and split into forward (CT) and reverse (GA) strands using a suitable utility. Each CT/GA .bed file was converted to .vcf format. CT/GA.vcf files were sorted using Picard SortVcf and the binarized DNA methylation (C/T) was inserted into genomic context using GATK FastaAlternateReferenceMaker function into CT/GA hg19 reference genomes made using the bismark bismark_genome_preparation command. Thus resulting in CT and GA .fasta references for each COI.

From each COI .bed file, contiguous genomic regions that were +/−31 bp of binarized DNA methylation values common across the 11 COI were selected using a shell script. Each COI CT/GA .fasta was sectioned into common contiguous regions using bedtools getfasta and all subset COI CT/GA .fasta files were combined to produce a .fasta file with 4999980 unique contigs (2272718 genomic regions×11 tissues×2 reference sequences [CT/GA]) from chromosomes 1-7, X & Y, constituting 20.8% of total cytosines within the hg19 reference genome. K-mers were indexed from the combined .fasta file using kallisto index −k 31, producing 1,035,106,323 k-mers.

Sequencing reads from WGBS cfDNA samples were assigned to COO using the kallisto quant function using the k-mer index described. The kallisto package utilises a process of pseudoalignment of k-mers. To reduce Type I errors, uniquely assigned reads to a single COO were selected using a suitable awk script. The number of uniquely assigned reads to each COO were counted using a suitable awk script. Note, due to the sex specificity of DNA methylation on the X and Y chromosomes, counts assigned to X/Y chr were removed from analysis.

As mentioned above, to determine the DNA methylation at a given cytosine position, a threshold value needs to be set based on fractional methylation. DNA methylation is largely cell-type specific. It was found that the bimodal distribution of DNA methylation strengthens as DNA methylation profiling is performed on more refined cell-populations, which reflects a population moving from more heterogeneous to more homogenous population.

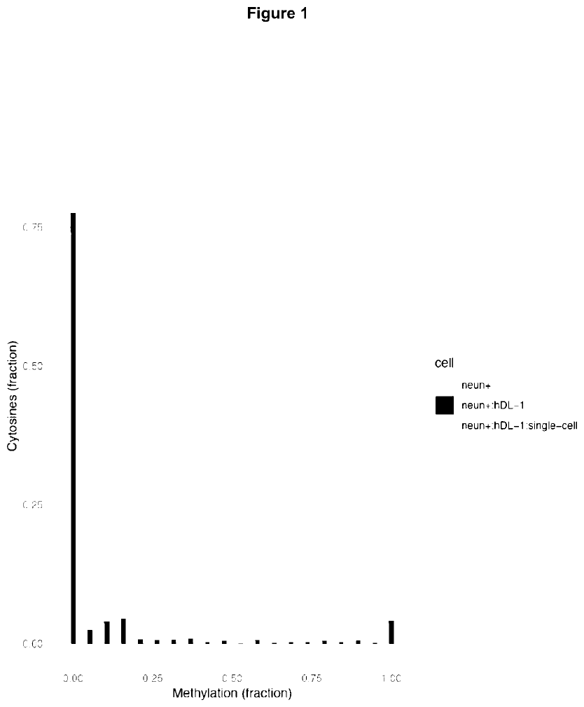

This is illustrated in FIG. 1, which shows the distribution of single-base resolution fractional DNA methylation calls (WGBS) of chromosome 21 with the Bimodal Coefficient (BC) strengthening as the cell population profiled is purified from broad cortical neurons (Lister R et al. (2013) Science, 341(6146): 1237905) (NeuN+, BC=0.885) to cortical layer specific neuron cell-type (NeuN+hDL-1, BC=0.907) to single cell-type (Luo C et al., (2017) Science, 357(6351): 600-604), in which DNA methylation is binary (NeuN+hDL-1 single-cell, BC=1). The thresholding value of 0.5 accounts for the majority of DNA methylation signals following sequencing of a purified cell-type in which DNA methylation fractional measurements>=0.5 are annotated “C”, whilst DNA methylation fractional measurements<0.5 are annotated “T”. The thresholding at a given position facilitates the binarization of the DNA sequence, where in this case, methylation fractional measurements greater than 0.5 indicate the presence of methylation at that given cytosine. Examination of other heterogeneous cell-populations of interest may require different threshold values depending on the heterogeneity of the cell's type.

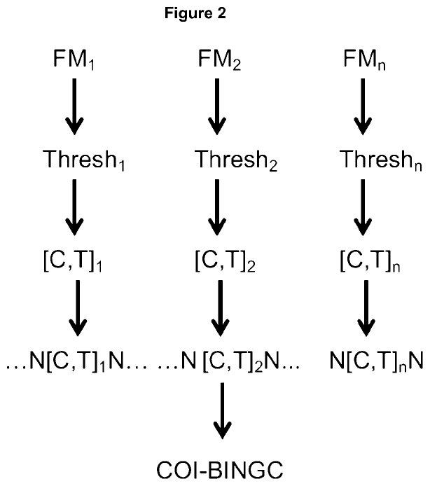

As shown in FIG. 2, k-mers are indexed from the binarized DNA methylation and inserted into Genomic Context (BINGC) for a COI. FIG. 2 shows n fractional methylations (FM₁ . . . FMₙ,). Each FM of cytosines for a COI are binarized into “C” or “T” [C,T] by reference to a predetermined threshold (Thresh₁ . . . Threshₙ). Each binarized DNA methylation value is then inserted into the genomic context. FIG. 2 shows such insertion as .N[C,T]ₙN. The COI-BINGC represents the binarized FM of all cytosines of a COI's genome.

This produces a reference sequence of binarised DNA methylation (binarised by the presence or absence of methylation at a particular position), mapped to specific positions (cytosines) within the genome of a COI. As each BINGC is derived from a COI, indexed k-mers are specific to a COI.

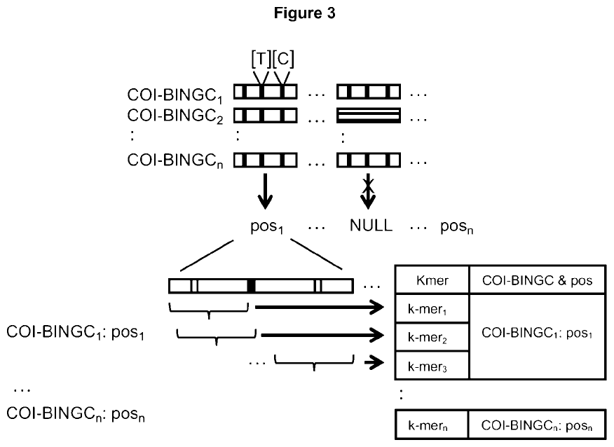

As shown in FIG. 3, sub-sequences of k-length (k-mer) are generated and indexed from each COI-BINGC reference sequence over a common position across all COI-BINGC. FIG. 3 shows contiguous genomic regions that are common across all COI-BINGC as defined as pos₁ (or posₙ). Where there are no binarised sequence data common to the COI-BINGCs (e.g. where COI-BINGC2 does not contain information for the region) this position is not used for further analysis (shown as NULL in FIG. 3).

FIG. 3 further shows the sequence over pose is broken up into k-mers and indexed for each COI-BINGO reference sequence. The indexed k-mers can then be used for subsequent hash table lookups of NGS reads from heterogenous DNA population samples, such as cfDNA, to annotate the COO of each DNA fragment. Unique k-mers are indexed for each given cell-type COI within contiguous genomic regions containing BINGO for all COI.

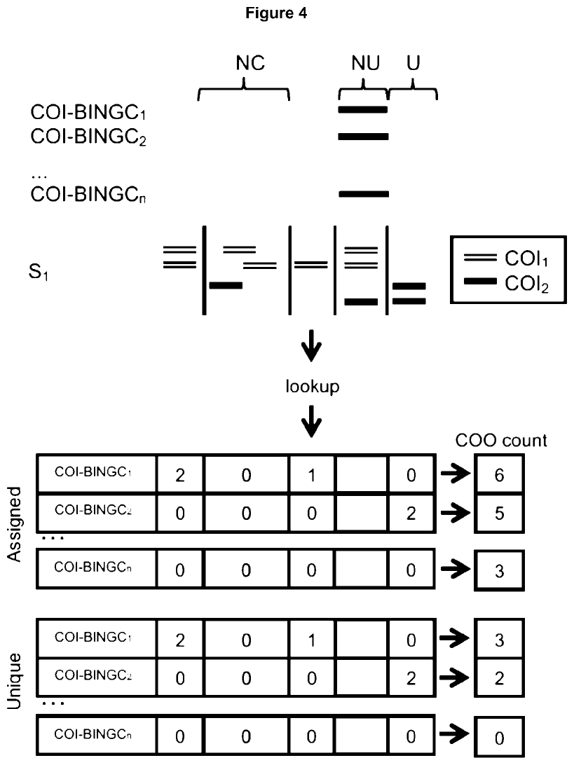

FIG. 4 illustrates the process of assigning cfDNA to a COO by using hash table lookup of all k-mers within a heterogeneous DNA molecule S₁ against the indexed table of all COI-BINGO databases. The sample sequence is broken up into k-mers, which are then used to look-up against each of the k-mers within each COI-BINGO database. The DNA molecules from the sample (S₁) are derived from multiple cells of interest (COI). Therefore, k-mers assigned to their respective COO through hash table lookup will be found to be either Unique to a COI-BINGO (designated U, in FIG. 4) or Non-Unique (NU). Regions with no coverage (NC) within one of the COI-BINGO (“NULL” in FIG. 3) are not considered.

K-mers from the sample sequence are looked-up and counted against COI-BINGC, whereby the total counts are “assigned” against COI-BINGO database. Reads from the S₁ sample that are uniquely assigned to a COI-BINGO are counted as “unique”. The unique COO counts are the unique assigned sequence reads to a COI by hash table lookup of the indexed k-mers. Unique DNA molecules (reads) are assigned to a single COI-BINGO having higher specificity in comparison to DNA molecules assigned to many COI-BINGO databases.

### Example 2—Identifying Cell-of-Origin Using cfDNA

cfDNA was extracted from 5 patients' CSF samples and analysed by WGBS. Sequencing libraries were prepared using Accel-NGS Methyl-Seq DNA Library Kit (Swift Biosciences, USA). In addition, 5 WGBS datasets were produced from Swift Biosciences from control peripheral blood plasma cfDNA. Libraries were made with the same library kit.

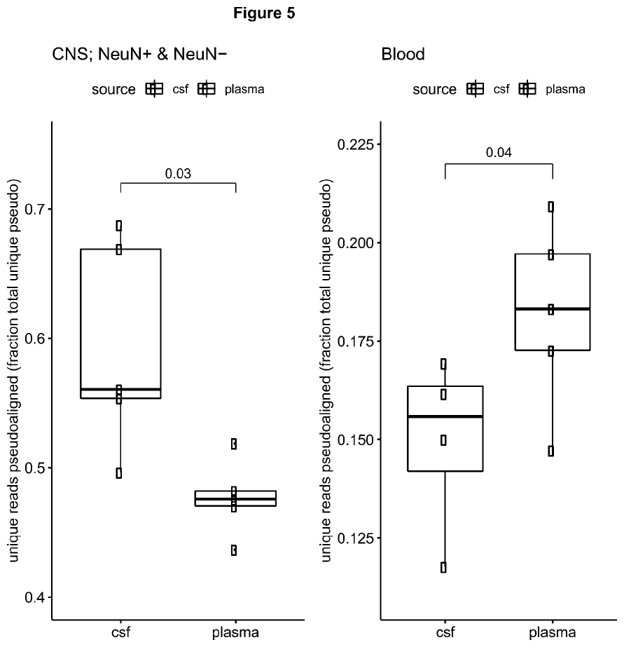

FIG. 5 is an analysis of assigning COO based on sequencing information produced from CSF-derived cfDNA versus blood-derived cfDNA, whereby the CSF-derived cfDNA was found to have a higher proportion of unique reads assigned to Central Nervous System cells (NeuN+ & NeuN−), whereas plasma had a higher proportion of cfDNA associated with blood cells. The Figure shows bisulphate-sequenced cfDNA from CSF or plasma (source shown in FIG. 5) assigned to a unique COO. Sequence reads from cfDNA derived from CSF were significantly (p=0.03) enriched for unique reads assigned to Central Nervous System cells (NeuN+ and NeuN−), whereas plasma-derived cfDNA had significantly (p=0.04) higher number of unique reads assigned to blood cells. The results are significant, as CSF from the brain would be expected to have higher amounts of brain-derived cfDNA in comparison to blood plasma, illustrating that the COI-BINGC followed by k-mer indexing/lookup can correctly assign DNA molecules from heterogenous DNA pools, such as cfDNA, to their respective COO. The result also supports the use of a liquid biopsy using cell-free DNA derived from CSF to detect somatic mosaicism in non-malignant brain diseases.

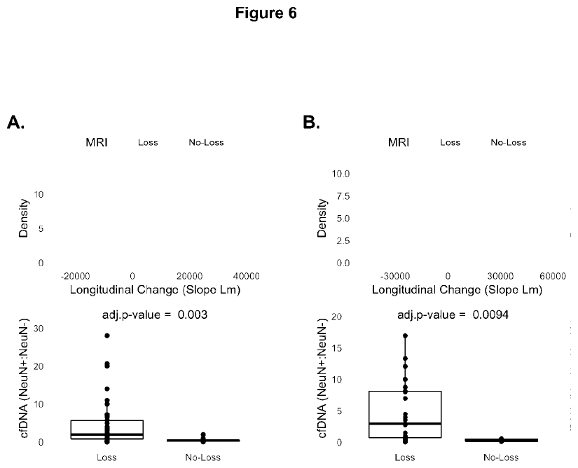

FIG. 6 illustrates the results of investigations into the presence of brain-derived cfDNA and cortical volume changes. A. Histogram (top) shows volume changes within the rostral anterior cingulate grey matter (left hemisphere) within behavioral variant Frontotemporal Dementia (FTD) patients (n=32). A significantly higher amount of brain-derived cfDNA (bottom) from cortical neurons was observed in samples taken from FTD patients exhibiting loss within the rostral anterior cingulate grey matter (B & H adjusted p-value=0.003). B Histogram (top) shows volume changes within the posterior cingulate (right hemisphere) grey matter within control patients (n=15). A significantly higher amount of brain-derived cfDNA from cortical neurons was observed in samples taken from Control patients exhibiting loss within the posterior cingulate grey matter (B & H adjusted p-value=0.0094). The results are significant as they show cortical loss of grey matter (quantified by longitudinal MRI) is associated with an increase in Neuron derived cfDNA.

Longitudinal blood biospecimens were sampled from 47 individuals diagnosed with FTD ([n=32; 59 samples]) or healthy controls (n=15; 29 samples). Plasma was extracted by centrifugation and cfDNA was extracted using the Analytik Jena PME cfDNA extraction kit (SE/SBS system) and bisulfite converted (Invitrogen MethylCode).

PCR amplification was performed using assays targeting genomic regions within Frontal Cortical Neuron (NeuN+) or Glial (NeuN−) with tissue-specific DNA methylation. Next Generation Sequencing libraries were generated by transposase mediated tagmentation (Illumina Nextera) and barcoding using Illumina sequencing adapters/indices. Sequencing was performed on the Illumina NextSeq500 (2×75 bp).

CT/GA FASTA references were made for COI (NeuN+/NeuN− and blood) for the targeted genomic regions and k-mers were indexed (kallisto index −k 31). Bisulfite sequencing reads (.fastq) were assigned to a COO (kallisto quant). A ratio of uniquely assigned reads to NeuN+: NeuN− were calculated for each plasma sample.

Cortical volumes from the 47 individuals were extracted (Freesurfer) from longitudinal T1-weighted MRI scans (2.7 ave scans/individual). Slopes derived from linear modeling of longitudinal cortical volumes (vol×yrs) were used as measurements of cortical loss/gain for each of 72 cortical regions of interest (DK atlas segmentations).

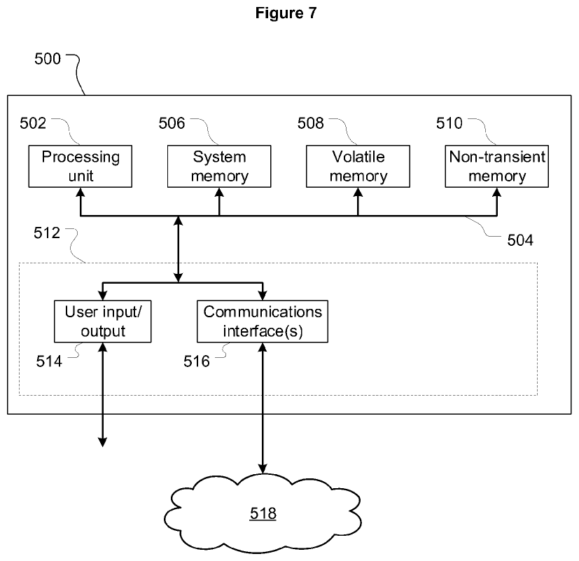

FIG. 7 provides a block diagram of a computer processing system 500 configurable to implement embodiments and/or features described herein, including by providing an execution platform and runtime environment for the above-mentioned sequencing applications. System 500 is a general purpose computer processing system. It will be appreciated that FIG. 16 does not illustrate all functional or physical components of a computer processing system. For example, no power supply or power supply interface has been depicted, however system 500 will either carry a power supply or be configured for connection to a power supply (or both). It will also be appreciated that the particular type of computer processing system will determine the appropriate hardware and architecture, and alternative computer processing systems suitable for implementing features of the present disclosure may have additional, alternative, or fewer components than those depicted.

Computer processing system 500 includes at least one processing unit 502—(for example a general or central processing unit, a graphics processing unit, or an alternative computational device). Computer processing system 500 may include a plurality of computer processing units. In some instances, where a computer processing system 500 is described as performing an operation or function all processing required to perform that operation or function will be performed by processing unit 502. In other instances, processing required to perform that operation or function may also be performed by remote processing devices accessible to and useable by (either in a shared or dedicated manner) system 500.

Through a communications bus 504, processing unit 502 is in data communication with a one or more computer readable storage devices which store instructions and/or data for controlling operation of the processing system 500. In this example system 500 includes a system memory 506 (e.g. a BIOS), volatile memory 508 (e.g. random access memory such as one or more DRAM modules), and non-volatile (or non-transitory) memory 510 (e.g. one or more hard disk or solid state drives). Such memory devices may also be referred to as computer readable storage media.

System 500 also includes one or more interfaces, indicated generally by 512, via which system 500 interfaces with various devices and/or networks. Generally speaking, other devices may be integral with system 500, or may be separate. Where a device is separate from system 500, connection between the device and system 500 may be via wired or wireless hardware and communication protocols, and may be a direct or an indirect (e.g. networked) connection.

Wired connection with other devices/networks may be by any appropriate standard or proprietary hardware and connectivity protocols, for example Universal Serial Bus (USB), eSATA, Thunderbolt, Ethernet, HDMI, and/or any other wired connection hardware/connectivity protocol.

Wireless connection with other devices/networks may similarly be by any appropriate standard or proprietary hardware and communications protocols, for example infrared, BlueTooth, WiFi; near field communications (NFC); Global System for Mobile Communications (GSM), Enhanced Data GSM Environment (EDGE), long term evolution (LTE), code division multiple access (CDMA—and/or variants thereof), and/or any other wireless hardware/connectivity protocol.

Generally speaking, and depending on the particular system in question, devices to which system 500 connects—whether by wired or wireless means—include one or more input/output devices (indicated generally by input/output device interface 514). Input devices are used to input data into system 500 for processing by the processing unit 502. Output devices allow data to be output by system 500. Example input/output devices are described below, however it will be appreciated that not all computer processing systems will include all mentioned devices, and that additional and alternative devices to those mentioned may well be used.

For example, system 500 may include or connect to one or more input devices by which information/data is input into (received by) system 500. Such input devices may include keyboards, mice, trackpads (and/or other touch/contact sensing devices, including touch screen displays), microphones, accelerometers, proximity sensors, GPS devices, touch sensors, and/or other input devices. System 500 may also include or connect to one or more output devices controlled by system 500 to output information.

Such output devices may include devices such as displays (e.g. cathode ray tube displays, liquid crystal displays, light emitting diode displays, plasma displays, touch screen displays), speakers, vibration modules, light emitting diodes/other lights, and other output devices. System 500 may also include or connect to devices which may act as both input and output devices, for example memory devices/computer readable media (e.g. hard drives, solid state drives, disk drives, compact flash cards, SD cards, and other memory/computer readable media devices) which system 500 can read data from and/or write data to, and touch screen displays which can both display (output) data and receive touch signals (input).

System 500 also includes one or more communications interfaces 516 for communication with a network, such as the Internet in environment 100. Via a communications interface 516 system 500 can communicate data to and receive data from networked devices, which may themselves be other computer processing systems.

System 500 stores or has access to computer applications (also referred to as software or programs)—i.e. computer readable instructions and data which, when executed by the processing unit 502, configure system 500 to receive, process, and output data. Instructions and data can be stored on non-transitory computer readable medium accessible to system 500. For example, instructions and data may be stored on non-transitory memory 510. Instructions and data may be transmitted to/received by system 500 via a data signal in a transmission channel enabled (for example) by a wired or wireless network connection over interface such as 512.

Applications accessible to system 500 will typically include an operating system application such as Microsoft Windows®, Apple OSX, Apple IOS, Android, Unix, or Linux.

In some cases part or all of a given computer-implemented method will be performed by system 500 itself, while in other cases processing may be performed by other devices in data communication with system 500.

1. A computer-implemented method for processing nucleic acid sequence data, the data including information on the methylation status of cytosine residues present in the sequence data, the method comprising the steps of:
assigning a binary methylation value selected from one of two possible values, to one or more of the cytosine residues;
extracting k-mers from the sequence data, each k-mer including a cytosine residue having one of the methylation values; and
storing the k-mers in a database.

2. A method according to claim 1, wherein the database includes a hash table, the method including the step of mapping each k-mer to a respective hash code, each hash code being suitable to address a storage location in the database.

3. A method according to claim 2, wherein the extracted k-mers are characteristic of the sequence data originating from a specified cell-of-interest, the method further including the steps of:
querying the database with a plurality input k-mers derived from an input sequence; and
returning a query result indicative of whether the input sequence originates from a cell-of-origin the same as the cell-of-interest.

4. A method according to claim 3, wherein the querying step comprises:
hashing one or more of the input k-mers to obtain a storage location address; and
comparing the input k-mer to the k-mer stored in the database at the storage location address.

5. A method according to claim 3, wherein the assigning step is performed by reference to a threshold.

6. A method according to claim 5, wherein the threshold is modified according to the specified cell-of-interest of the sequence data.

7. A method according to claim 3, wherein the specified cell-of-interest is a cell type selected from the group consisting of a pancreatic beta cell, a pancreatic exocrine cell, a hepatocyte, a brain cell, a lung cell, a uterus cell, a kidney cell, a breast cell, an adipocyte, a colon cell, a rectum cell, a cardiomyocyte, a skeletal muscle cell, a prostate cell and a thyroid cell.

8. A method according to claim 3, wherein the specified cell-of-interest is from a tissue selected from the group consisting of pancreatic tissue, liver tissue, lung tissue, brain tissue, uterus tissue, renal tissue, breast tissue, fat, colon tissue, rectum tissue, heart tissue, skeletal muscle tissue, prostate tissue and thyroid tissue.

9. A method according to claim 3, wherein the specified cell-of-interest is a cancer cell, tumour cell or transformed cell.

10. A method according to claim 2, wherein the extracted k-mers are characteristic of the sequence data originating from a virus.

11. A computer-readable storage medium having instructions encoded thereon which when executed by a processor, cause the processor to process nucleic acid sequence data, the data including information on the methylation status of cytosine residues present in the sequence data, by:
assigning a binary methylation value selected from one of two possible values, to one or more of the cytosine residues;
extracting k-mers from the sequence data, each k-mer including a cytosine residue having one of the methylation values; and
storing the k-mers in a database.

12. A medium according to claim 11, wherein the database includes a hash table, wherein the instructions further cause the processor to map each k-mer to a respective hash code, each hash code being suitable to address a storage location in the database.

13. A medium according to claim 12, wherein the extracted k-mers are characteristic of the sequence data originating from a specified cell-of-interest, wherein the instructions further cause the processor to:
query the database with a plurality input k-mers derived from an input sequence; and
return a query result indicative of whether the input sequence originates from a cell-of-origin the same as the cell-of-interest.

14. A medium according to claim 13, wherein the querying step comprises:
hashing one or more of the input k-mers to obtain a storage location address; and
comparing the input k-mer to the k-mer stored in the database at the storage location address.

15. A medium according to claim 14, wherein the assigning step is performed by reference to a threshold.

16. A medium according to claim 15, wherein the threshold is modified according to the specified cell-of-interest of the sequence data.

17. A medium according to claim 13, wherein the specified cell-of-interest is a cell type selected from the group consisting of a pancreatic beta cell, a pancreatic exocrine cell, a hepatocyte, a brain cell, a lung cell, a uterus cell, a kidney cell, a breast cell, an adipocyte, a colon cell, a rectum cell, a cardiomyocyte, a skeletal muscle cell, a prostate cell and a thyroid cell.

18. A method according to claim 13, wherein the specified cell-of-interest is from a tissue selected from the group consisting of pancreatic tissue, liver tissue, lung tissue, brain tissue, uterus tissue, renal tissue, breast tissue, fat, colon tissue, rectum tissue, heart tissue, skeletal muscle tissue, prostate tissue and thyroid tissue.

19. A method according to claim 13, wherein the specified cell-of-interest is a cancer cell, tumour cell or transformed cell.

20. A method according to claim 12, wherein the extracted k-mers are characteristic of the sequence data originating from a virus.

21. A method of generating a library of polynucleotide subsequences representative of one or more cells of origin, the method comprising:
a) providing a plurality of DNA methylation profiles for a genomic sequence for a cell of origin;
b) annotating the genome sequence of the cell of origin at each cytosine residue, based on the DNA methylation profiles, to create binarised reference genomic information for the cell of origin;
c) repeating steps a) and b) for one or more additional cells of origin;
d) generating a series of subsequences of length k (k-mers) from the binarised reference genomic information from the different cells of origin,
thereby generating a library of polynucleotide subsequences representative of one or more cells of origin.

22. A method according to claim 21, wherein the step of annotating the genome sequence of the cell of origin comprises i) binarising each cytosine nucleotide in the genome sequence, and ii) inserting the binary value assigned to each cytosine during the binarising into the genomic context of the cytosine nucleotide, to create sense and antisense genomic DNA fragments comprising information on the methylation status of each cytosine nucleotide.

23. A method according to claim 22, wherein the binary values comprise:
“methylated” or “not methylated”; “C” or “T”, or two other values representing the methylation state of each cytosine.

24. A computer-implemented method of processing genome sequence data for a cell of origin, the method comprising:
a) annotating the genome sequence data at each cytosine residue, based on a DNA methylation profile applicable to the cell of origin, to create binarised reference genomic information for the cell of origin;
b) repeating step a) for one or more additional cells of origin;
c) generating a series of subsequences of length k (k-mers) from the binarised reference genomic information from the different cells of origin; and
d) storing the k-mers in a cell-of-origin database for each cell of origin to thereby generate a library of polynucleotide subsequences representative of one or more cells of origin.

25. A computer-readable storage medium having instructions encoded thereon which when executed by a processor, cause the processor to process genome sequence data for a cell of origin, by:
a) annotating the genome sequence data at each cytosine residue, based on a DNA methylation profile applicable to the cell of origin, to create binarised reference genomic information for the cell of origin;
b) repeating step a) for one or more additional cells of origin;
c) generating a series of subsequences of length k (k-mers) from the binarised reference genomic information from the different cells of origin; and
d) storing the k-mers in a cell-of-origin database for each cell of origin to thereby generate a library of polynucleotide subsequences representative of one or more cells of origin.

26. A method of determining a tissue or cell of origin of a nucleic acid sequence of unknown origin, the nucleic acid sequence including information on the methylation status of cytosine residues present in the nucleic acid sequence, the method comprising:
providing or having obtained a library or database of subsequences representative of one or more cells of origin, produced by performing the method according to any one of claim 1, 21 or 24;
dividing the nucleic acid sequence into one or more subsequences;
querying the database of subsequences with the subsequences; and
receiving a result of the query that indicates the tissue or cell of origin of the nucleic acid.

27. A method of characterizing a cfDNA sample from a subject, comprising:
receiving a plurality of sequencing reads for a cfDNA sample from a subject, wherein each sequencing read comprises methylation sequencing data obtained from a consecutive nucleic acid sequence of 25 or more nucleic acids; and
querying a library or database produced by performing the method according to any one of claim 1, 21 or 24 with the plurality of sequencing reads to compute one or more likelihood scores, wherein the likelihood score is indicative of the likelihood that the sequences in the cfDNA sample correspond to sequences from a given cell-of-origin.

28. A method of determining the likelihood that an individual is suffering from a disease or condition characterised by cell death in an organ or cell of interest, the method comprising:
providing an individual for whom diagnosis of a disease or condition characterised by cell death in an organ or cell of interest is required;
providing or having obtained a query nucleic acid sequence of unknown origin, obtained from a sample of plasma-derived or CSF-derived cfDNA from the individual;
providing of having obtained a database of subsequences representative of one or more cells of origin produced by performing the method according to any one of claim 1, 21 or 24, wherein the one or more cells of origin comprises cells of one or more organs, organ tissues or cell of interest;
determining that the individual is likely suffering from a condition or disease characterised by cell death in an organ when subsequences of the query nucleic acid coincide with subsequences in the database representative of cells of an organ, organ tissues or cell of interest; or
determining that the individual is likely not suffering from a condition or disease characterised by cell death in an organ when one or more subsequences of the query nucleic acid sequence do not coincide with subsequences in the database representative of cells of an organ, organ tissues or cell of interest.

29. A method according to claim 28, wherein the disease or condition characterised by cell death in an organ or tissue is a neurodegenerative disorder, a disorder of the thyroid gland or a kidney disorder.

30. A method of determining the likelihood that an individual is suffering from a neurodegenerative disease or condition, the method comprising:
providing an individual for whom diagnosis of a neurodegenerative disease or condition is required;
providing or having obtained a query nucleic acid sequence of unknown origin, obtained from a sample of plasma-derived cfDNA from the individual;
providing or having obtained a database of subsequences representative of one or more cells of origin produced by performing the method according to any one of claim 1, 21 or 24, wherein the one or more cells of origin comprises cells of neurological origin;
determining that the individual is likely suffering from a neurodegenerative disease or condition when one or more subsequences of the query nucleic acid sequence coincide with subsequences in the database representative of cells of neurological origin; or
determining that the individual is likely not suffering from a neurodegenerative disease or condition when one or more subsequences of the query nucleic acid sequence do not coincide with subsequences in the database representative of cells of neuronal origin.

31. A method of detecting the presence of DNA from a cell or tissue of origin and identifying if a subject has a disease or condition characterised by necrosis, apoptosis or other mode of death of the cell or tissue, the method comprising:
receiving sequencing data of cell-free methylated DNA from a test sample obtained from a subject suspected of having, or at risk of having a disease or condition characterised by necrosis, apoptosis or other mode of death of the cell or tissue;
comparing subsequences of the cell-free methylated DNA to a library or database produced by performing the method according to any one of claim 1, 21 or 24;
identifying that the subject has a disease or condition characterised by necrosis, apoptosis or other mode of death of the cell or tissue when one or more compared subsequences coincide with subsequences present in the library or database; or
identifying that the subject does not have a disease or condition characterised by necrosis, apoptosis or other mode of death of the cell or tissue when one or more compared subsequences do not coincide with subsequences present in the library or database.

32. A method according to any one of claims 26 to 31, wherein the nuclucleic acid sequence is obtained from one of whole genome bisulfite sequencing, TET-assisted pyridine borane sequencing (TAPS), Third Generation Sequencing or targeted DNA methylation sequencing.

33. A computer-implemented method of determining the cell or tissue of origin for cfDNA obtained from a subject, the method comprising:
receiving, at at least one processor, sequencing data of cell-free methylated DNA from a subject sample;
comparing, at the at least one processor, subsequences of the sequencing data to reference database of cell-free methylated DNA subsequences from healthy and cancerous individuals;
identifying, at the at least one processor, that one or more of the compared subsequences coincides with one or more of the cancerous cell-free methylated DNA subsequences comprised in the reference cell-free methylated DNA subsequences.
